(19) United States
(12) Patent Application Publication
(10) Pub. No.: US 2010/0069673 A1
(43) Pub. Date: Mar. 18, 2010

(54) Neutralization of Ethylenediamine Hydrochloride and recovery of Ethylenediamine

(76) Inventors: Romas Cesas, Naperville, IL (US); Theodore Zajack, Alsip, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/283,679

(22) Filed: Sep. 15, 2008

(51) Int. Cl. C07C 209/82

(52) U.S. Cl. 564/498

(57) ABSTRACT

In a neutralization and recovery process ethylene diamine hydrochloride (EDA-HCL) is neutralized and ethylene diamine (EDA) is recovered using a mixture of solid caustic dissolved in a non-aqueous solvent, for example an alcohol.

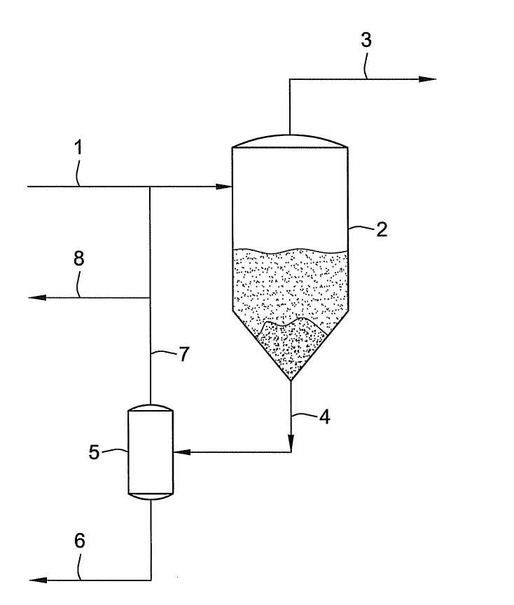

### FIELD OF INVENTION

The present invention provides a process to recover ethylenediamine (EDA) from a byproduct or waste stream comprising ethylenediamine hydrochloride (EDA-HCl) without the addition of aqueous neutralizing reagent. A solid caustic reagent is dissolved in an alcohol and added to the byproduct or waste stream which neutralizes the EDA-HCl forming EDA and a salt precipitate. Manipulation of pressure and temperature allows for the recovery of the EDA.

### BACKGROUND OF THE INVENTION

Neutralization of ethylenediamine hydrochloride to recover ethylenediamine is known to the art, including the use of aqueous caustic solutions to neutralize the EDA-HCl by the following equation:

EDA-HCl+NaOH aq (50%)→NaCl+EDA+H₂O

These processes that used aqueous caustic solutions unfortunately added significant quanties of water to the water formed during the reaction, thus making the recovery of the EDA more difficult and energy intensive. Other processes tried to avoid adding excess water to the overall recovery process by using sodium methoxide as the neutralizing reagent, however, such this reagent is expensive making the process economics unfavorable. As such, there exists a need to recover EDA from an EDA-HCl containing feedstock that uses an inexpensive reagent and that is energy efficient. Our invention solves this problem by using a solid caustic reagent dissolved in a organic solvent that avoids introducing water into the recovery process. These and other advantages will become evident from the following more detailed description of the invention.

### SUMMARY OF THE INVENTION

Our invention is directed to a process for neutralizing ethylene diamine hydrochloride (EDA-HCl) and recovering ethylene diamine (EDA) without using an aqueous reagent to neutralize the EDA-HCl. Our process involves the steps of first providing at least one reactor capable of maintaining pressures above and below atmospheric. It is, however, within the scope of our invention to use one or more swing reactors to simulate a continuous process. More preferably, a stirred tank reactor provides a convenient vessel to perform our process. Allow the following descriptions will relate to a batch type operation, one skilled in the art readily understands that our invention could be performed as a semi-continuous of continuous operation.

The first step in our process involves determining the amount of EDA-HCL contained in the byproduct or waste solution to be neutralized. Once this is determined, a determination is made of at least a stoichiometric amount of solid caustic, preferably NaOH, KOH, or a mixture thereof, that will react with the EDA-HCl to form EDA according to the following reaction.

EDA-HCl+Caustic_{solid}→Caustic Salt_{precipitate}+EDA+H₂O

It is preferred to first dissolve the calculated solid caustic in an alcohol or equivalent organic solvent prior to addition of the feed. It is preferred to select an alcohol especially when alcohol is present as a byproduct in the feed to be treated. Any alcohol that can dissolve the solid caustic can be used. Preferred alcohols include methanol, ethanol, or a mixture thereof. The amount of alcohol mixed with the solid caustic added is determined by using the reported solubility of the caustic used in the specific alcohol used (e.g. 1 gm of NaOH dissolves in 4.2 ml of methanol). The alcohol is heated to 50° C. to enhance the rate of solubility of the caustic in the alcohol. Once the alcohol and caustic are mixed to form a solution, the feed compromising a mixture of ethylene diamine hydrochloride (EDA-HCL) and ethylene diamine (EDA) is added to the reactor. Mixing causes the above reaction to form EDA, water and a precipitate containing a solid salt. The precipitated salt is continuously removed from the reactor bottom during the course of the entire process. Any number of known methods to remove the salt precipitate can be used, such as gravity settling, filtering, evaporation, or crystallizer, however, centrifugation is the preferred method. In some cases supernatant from the salt separation is returned to the reactor, especially if it contains EDA or un-reacted EDA-HCl. The mixture of feed, alcohol and caustic is then heated to at least 65.5° C., preferably to temperature in the range of from about 65.5° C. to about 110° C. This heating step causes the organic solvent (in the preferred case an alcohol) to vaporize and be removed as an overhead from the reactor mixture. The temperature of the solution mixture in the pot will rise as the alcohol is removed.

Once the majority of the alcohol is removed, the pressure and temperature of the reactor are increased to break the azeotrope that forms between water and EDA, thus allowing the water formed during the neutralization reaction to be distilled off and removed from the reactor. Preferably the pressure will be increased to a maximum of 3 atmospheres, preferably in the range of from about 2.8 atm to about 3.2 atm and the temperature will be increased to a maximum of about 180° C., preferably in the range of from about 170° C. to about 190° C. The increased temperature and pressure will be maintained until an analysis of the distilled water shows EDA present. Preferably, once the EDA content in the distilled water reaches about 1 wt. % then the pressure of the reactor is reduced to about atmospheric, preferably less than atmospheric, most preferably around 200 mm Hg. At this point the overhead distilled product from the reactor will be dry EDA, defined as <0.3 wt. % H₂O. The solution mixture in the pot is then cooled to below the boiling point expected at the reduced pressure of approximetly 200 mm Hg (˜78° C.) before heat is applied to begin distillation.

These and other embodiments will become more apparent from the detail description of the preferred embodiment contained below.

### BRIEF DESCRIPTION OF THE DRAWING

The accompanying FIGURE schematically illustrates a one possible embodiment for a batch operation of the process according to our invention.

### DETAILED DESCRIPTION OF A PREFERRED EMBODIMENT

As mentioned, our process can be a batch, semi-continuous or continuous operation. The following embodiment is directed to a batch operation using a single reactor, preferably a stirred tank reactor even though the means for mixing is not shown. Referring now to the drawing, methanol is added through line 1 to reactor 2 and then solid NaOH is added. Mixing occurs until the caustic is dissolved. Alternatively, the caustic and methanol could be mixed in a separate vessel and added as a solution through line 1. The feed mix of EDA-HCl and EDA is added next through line 1 and mixed with the caustic-methanol mixture. As the neutralization reaction of the caustic and EDA-HCl takes place a precipitate of NaCl forms and is continuously removed from the bottom of reactor 2 through line 4 and fed to centrifuge 5, where the solid NaCl is removed via line 6 and supernatant is removed via line 7. Depending on composition of the supernatant, it can all be returned to the process or a portion can be continuously removed via line 8. As temperature of the mixture in reactor 2 is increased the methanol begins to distill and is removed via line 3. Later in the process water is likewise removed as a vapor via line 3 and eventually recovered dry EDA product is removed as an overhead in line 3.

The foregoing description of the specific embodiments will so fully reveal the general nature of the invention that others can, by applying current knowledge, readily modify and/or adapt for various applications such specific embodiments without departing from the generic concept, and therefore such adaptations and modifications are intended to be comprehended within the meaning and range of equivalents of the disclosed embodiments. It is to be understood that the phraseology or terminology herein is for the purpose of description and not of limitation.

The means, materials, and steps for carrying out various disclosed functions may take a variety of alternative forms without departing from the invention. Thus, the expressions “means to . . . ” and “means for . . . ”, or any method step language as may be found in the specification above or the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same function can be used; and it is intended that such expressions be given their broadest interpretation within the terms of the following claims.

We claim:

1. A process for neutralizing ethylene diamine hydrochloride and recovering ethylene diamine comprising, in combination, the steps,
a. providing at least one reactor capable of pressurization above atmospheric;
b. providing a known quantity of feed compromising a mixture of ethylene diamine hydrochloride (EDA-HCL) and ethylene diamine (EDA);
c. determining the concentration of EDA-HCL contained in the feed;
d. adding alcohol to the reactor;
e. adding at least a stoichiometric amount of solid caustic to the reactor sufficient to neutralize the EDA-HCL determined in step c.;
f. mixing the caustic and alcohol until the solid caustic has dissolved;
g. charging the feed to the reactor;
h. mixing the feed with the alcohol and caustic mixture to form EDA, water and a precipitate containing a solid salt;
i. continuously removing the precipitated salt from the reactor;
j. heating the mixture in step h. to at least 65.5° C. to vaporize the alcohol;
k. removing the alcohol vapor from the reactor;
l. increasing the pressure and temperature in the reactor to distill the water from the EDA and the reactor vessel;
m. reducing the pressure of the reactor when EDA is detected in the removed distilled water; and
n. removing dry EDA from the reactor.

2. The process of claim 1 wherein the solid caustic is NaOH and the precipitated salt is NaCl.

3. The process of claim 1 wherein the solid caustic is KOH and the precipitated salt is KCl.

4. The process of claim 1 wherein the precipitated salt is removed via centrifugation.

5. The process of claim 1 wherein the alcohol is selected from the group consisting of ethanol, methanol or mixtures thereof.

6. The process of claim 1 wherein the pressure is increased from atmospheric to 3 atmospheres or less.

7. The process of claim 1 wherein the pressure is reduced when the amount of EDA detected in the distilled water reaches approximately 1 wt. %.

8. The process of claim 7 wherein the pressure is reduced to atmospheric or below.
